(19) United States
(12) Patent Application Publication
(10) Pub. No.: US 2013/0004045 A1
(43) Pub. Date: Jan. 3, 2013

(54) SYSTEM AND METHOD FOR 3D TOMOGRAPHIC IMAGE RECONSTRUCTION IN THE CIRCULAR GEOMETRY

(76) Inventor: Frank Dennerlein, Forchheim (DE)

(21) Appl. No.: 13/540,607

(22) Filed: Jul. 2, 2012

Related U.S. Application Data
(60) Provisional application No. 61/503,652, filed on Jul. 1, 2011.

(51) Int. Cl. G06K 9/00; A61B 6/03

(52) U.S. Cl. 382/131; 378/19

(57) ABSTRACT

A technique for 3D tomographic image reconstruction in the circular geometry (e.g., cone-based circular geometry) is disclosed. The technique may include data filtering using an initial 2D Laplace operation and a subsequent, non-local 2D filtering operation. The first filtering step thus only acts locally on the data so that it can be carried out accurately even in presence of (transaxial) data truncation. This feature may provide increased flexibility with respect to truncated projections as compared with certain standard FBP methods. Simulation studies show that the technique yields, for heavily transaxially-truncated data, an image quality that is similar to that obtained with the Feldkamp method applied with an explicit extrapolation scheme.

### CROSS-REFERENCE TO RELATED APPLICATIONS

This application claims priority to U.S. Provisional Patent Application No. 61/503,652 filed Jul. 1, 2011. The contents of which is incorporated herein by reference in its entirety.

### TECHNICAL FIELD

This disclosure relates to tomographic imaging, e.g., for use in an X-ray system. For example, the disclosure relates to filtered-backprojection (FBP) algorithms for 3D reconstruction in the circular geometry.

### BACKGROUND

Cone-beam X-ray computed tomography (CB-CT) has become a widely-used imaging technology with various applications in the medical field, in the field of non-destructive testing and in the security sector. Practically-available flat-panel volumetric CT scanners can provide 3D images of the investigated object that are computed from a series of 2D projection images obtained during a single rotation of the system around the stationary object. In most of these flat-panel CT systems, reconstruction is carried out using analytical filtered-backprojection (FBP) algorithms, such as the Feldkamp algorithm. These algorithms are both efficient and robust and also yield high image quality in the practical application.

Achieving high image quality becomes more difficult, however, when projections are truncated, particularly when only a portion of the entire width of the object was captured during acquisition. Truncated projections can be caused by limitations in the detector size, but also by a narrow collimation of the X-ray beam just to the region of interest. The straight-forward application of a filtered-backprojection method on truncated data typically causes inacceptable high-intensity artifact structures in the reconstruction results close to the boundaries of the field-of-view.

While some specific scenarios of truncation still allow accurate reconstruction using dedicated analytical reconstruction methods, other truncation cases can only be tackled in practice using approximate methods. The most common approach is to try to extrapolate in each projection image the measured values explicitly beyond the image boundaries in case of truncation and to perform conventional reconstruction from the extrapolated projections.

### A. Geometry and Notation

This disclosure utilizes the planar, circular acquisition geometry with a flat detector, as displayed in FIG. 1. In particular, FIG. 1 shows a 3D circular cone-beam acquisition geometry with a flat detector.

We denote the spatial distribution of the object density with the function ƒ(x) where x=(x, y, z) and assume that the X-ray source moves along the trajectory a(λ)=(R cos λ, R sin λ, 0) during the scan. The quantity λ then corresponds to the polar angle of the source and R to the scan radius. Data is acquired with a planar 2D detector that is parallel to the vectors eᵤ(λ)=(−sin λ, cos λ, 0) and eᵥ(λ)=(0,0,1), orthogonal to e_{w}(λ)=(cos λ, sin λ, 0) and at distance D from the source. Points on the detector are specified using u=(u,v), where u and v are coordinates measured along the axes eᵤ(λ) and eᵥ(λ) respectively. The point u=(0,0) is set to the orthogonal projection of a(λ) onto the detector. We assume that a rectangular detector is used that measures radiation on all points uεD with D=[−uₘ, uₘ]×[−vₘ, vₘ].

Data acquisition in this geometry then yields a function

g(λ,u,v)=∫₀^{∞}ƒ(a(λ)+t\hat{α}(λ,u,v))dt (1)

where α(λ,u,v) is the unit direction vector of the ray connecting α(λ) with the point u on the detector plane. CB data at fixed λ and with uεD will here be called one projection image.

For each λ, we can determine the set Ω_{λ}={u|g(λ,u,v)≠0} that describes the shadow of the object on the detector plane for the projection λ, as shown in FIG. 2. In particular, FIG. 2 illustrates the 2D flat panel detector. An arbitrary line on the detector (e.g., indicated in bold) can be parameterized using the angle μ between the line normal vector and the u-axis and the distance |s| between the line and the origin u=(0,0). If Ω_{λ}εD, we will call the projection image non-truncated. If Ω_{λ} exceeds the area D (along u), we will call the projection (transaxially) truncated.

Furthermore, let us use h_{R}(•) and h_{H}(•) to denote the spatial representation of the ramp filter kernel and the Hilbert filter kernel, respectively.

### B. Feldkamp Reconstruction

The Feldkamp algorithm is a popular method for tomographic image reconstruction in the circular CB geometry described above. Feldkamp's method computes an estimate ƒ^{(FDK)}(x) of the object function ƒ(x) by backprojecting filtered projection data g_{F} according to

$${f}^{( FDK )} ( \underline{x} ) = {\int }_{{λ}_{1}}^{{λ}_{2}} \frac{RD}{{[ R - \underline{x} \cdot {\underline{e}}_{w} ( λ ) ]}^{2}} {g}_{F} ( λ , {u}^{*} , {v}^{*} ) d λ . \qquad ( 2 )$$

Here, u* and v* describe the coordinates of the CB projection of x onto the detector and g_{F} is defined for transaxially non-truncated data as

g_{F}(λ,u,v)=∫_{-∞}^{∞}h_{F}(u−u′)g₁(λ,u′,v)du′ (3)

The function g₁ corresponds to the weighted CB data

$${g}_{1} ( λ , u , v ) = \frac{Dm ( λ , u )}{\sqrt{{D}^{2} + {u}^{2} + {v}^{2}}} g ( λ , u , v ) . \qquad ( 4 )$$

For reconstruction from a full circular scan, the function m is given as m(λ, u)=0.5. For reconstruction from a partial circular scan, m(λ, u) is usually set heuristically to a Parker-like weighting function to approximately equalize the redundancies in the CB data set.

Note that the convolution in (3) is not affected by axial truncation, but that the Feldkamp filter operation applied on the measured data inside D only allows us to compute

g_{F}^{(FDK)}(λ,u,v)=∫_{|u|≦u}_{ₘ}h_{F}(u−u′)g₁(λ,u′,v)du′ (5)

The difference between this computable g_{F}^{(FDK)} and the function g_{F} as defined in (3) is given as

ε(λ,u,v)=∫_{|u|>u}_{ₘ}h_{F}(u−u′)g₁(λ,u′,v)du′ (6)

and becomes 0 only along the lines v₀ for which g₁ (λ, u, v₀)=0 for all |u|>uₘ. Because the distribution of values of g₁ inside Ω_{λ} typically strongly deviates from 0, however, this condition is usually significantly violated in presence of transaxial data truncation. Because of this and because of the infinite support of h_{F}(u), transaxial data truncation along a line vₜᵣᵤₙₑ prohibits a correct computation of g_{F} along this entire line, and thus creates truncation artifacts in the reconstructions.

A common way to reduce truncation artifacts is to explicitly extrapolate the measured CB data prior to the filtering operation. This requires an estimation of the values of g(λ,u,v) at points that are inside Ω_{λ} but outside D. Several data extrapolation schemes have been suggested that produce satisfying results, but all these methods require an additional heuristic data extrapolation step.

### SUMMARY

In one embodiment, a method for performing a 3D tomographic image reconstruction in the circular geometry includes receiving cone-based (CB) projection data associated with an object; applying a weighting function to the received data; performing a localized filtering of the data using a 2D Laplace operation; performing a non-localized filtering of the data using a 2D Radon-based filtering operation; and performing a 3D Cone-beam backprojection of the data to generate a tomographic image.

In another embodiment, a system for performing a 3D tomographic image reconstruction in the circular geometry includes an X-ray source; an X-ray detector configured to detect radiation from the X-ray source to collect cone-based (CB) projection data associated with an object; and a processing circuit configured to generate a tomographic image of the object by applying a weighting function to the received data, performing a localized filtering of the data using a 2D Laplace operation, performing a non-localized filtering of the data using a 2D Radon-based filtering operation, and performing a 3D Cone-beam backprojection of the data to generate the tomographic image of the object.

### BRIEF DESCRIPTION OF THE DRAWINGS

Example embodiments will be explained in more detail below with reference to figures, in which:

FIG. 1 illustrates a 3D circular cone-beam acquisition geometry with a flat detector.

FIG. 2 illustrates a 2D flat panel detector in the 3D circular cone-beam acquisition geometry.

FIG. 3 illustrates results of an example Experiment A, showing slices through a short-scan reconstruction (240 degrees) obtained from non-truncated CB data using Feldkamp (left) and the method disclosed herein (right), visualized in the window [0, 100] HU. Top images: slice z=0 mm; bottom images: slice z=30 mm.

FIG. 4 illustrates results of an example Experiment B: Reconstruction from severally truncated, full-scan CB data, for Feldkamp (top) Feldkamp with constant extrapolation (center) and the method disclosed herein (bottom). The filtered projection images at λ=0 and with uεD (left) and the reconstruction slices at z=0 mm in the window of width 800 units on the Hounsfield scale (right).

FIG. 5 illustrates an example C-arm X-ray imaging system configured or programmed to implement the method disclosed herein, according to one embodiment.

FIG. 6 illustrates an X-ray source and detector of the example system of FIG. 3, being configured to rotate around the subject, according to one embodiment.

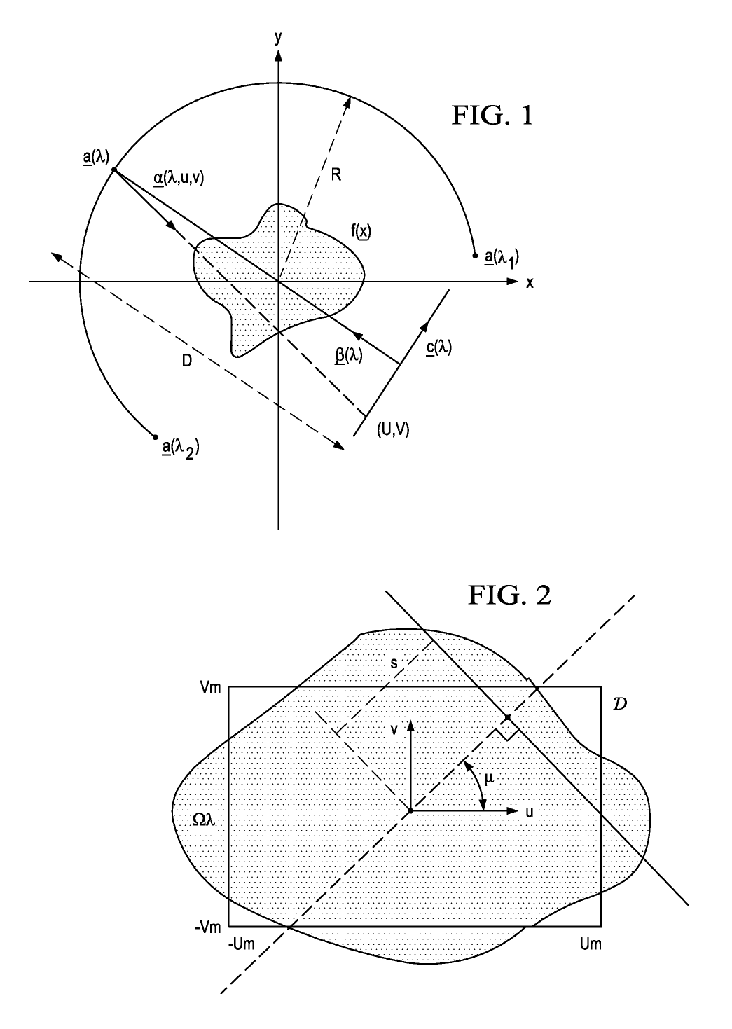

### DETAILED DESCRIPTION

Some embodiments provide a CB reconstruction algorithm that achieves similar results as the extrapolation methods in case of data truncation, but that does not require an explicit extrapolation of projection data. Instead, the algorithm disclosed below implicitly provides a greater flexibility to truncation than conventional filtered-backprojection algorithms. Sections A. Modification of Filter Operation and B. Alternative 2D Radon inversion below describe concepts used during that derivation; and section C. 3D Cone-beam Reconstruction Formula below discloses an example algorithm in total, according to one embodiment.

### A. Modification of Filter Operation

The reconstruction method disclosed herein follows the scheme of equations (2) and (4) above, but uses an alternative method to achieve the filtering defined in (3). Let us for simplicity focus on the single CB projection at λ=λ₀ only and assume that this projection is non-truncated. The outcome of ID ramp filtering (in u) of this weighted projection g₁ can also be obtained through the following 2D operations:

Step 1: Computation of the 2D Radon Transform of g₁ using the Notation Defined in the Caption of FIG. 2 Over the Range

$$s ∈ [ - ∞ , ∞ ] and μ ∈ [ 0 , π ) \\ {p}_{1} ( μ , s ) = {\int \int }_{\underline{u} ∈ {Ω}_{λ}} {g}_{1} ( {λ}_{0} , u , v ) δ ( \underline{u} \cdot ( \begin{matrix}cos μ \\ sin μ\end{matrix} ) - s ) d \underline{u} . \qquad ( 7 )$$

Step 2: Differentiation of p₁ with Respect to s

$${p}_{2} ( μ , s ) = \frac{\partial }{\partial s} {p}_{1} ( μ , s ) \qquad ( 8 )$$

Step 3: Weighting of p₂

$${p}_{3} ( μ , s ) = - \frac{1}{2 π} \frac{R}{D} \vert cos μ \vert {p}_{2} ( μ , s ) \qquad ( 9 )$$

Step 4: Convolution of P₃ with the Hilbert Kernel h_{H} (s)

p₄(μ,s)=∫_{-∞}^{∞}h_{H}(s−s′)p₃(μ,s′)ds′ (10)

Step 5: Computing the Inverse 2D Radon Transform of p₄

g_{F}(λ₀,u,v)=p₅(u,v)= [symbol] {p₄(μ,s)}. (11)

The operator R⁻¹ in (10) denotes the inverse of the 2D Radon transform in parameters μ, s. This operation could be implemented using the classical, ramp-filtered FBP algorithm. Here, however, we apply a different 2D Radon inversion formula that allows simplification of some steps listed above.

### B. Alternative 2D Radon Inversion

This section discloses an algorithm for the inverse 2D Radon transform operator R⁻¹ that occurs in step 5 of the previous section. For convenience, let p(μ, s):=p₄(μ, s) be the intermediate filter result defined in (9). Let furthermore p_{F}(μ, s) and p_{H}(μ, s) denote the outcomes of the ID convolutions of p(μ, s) with the ramp kernel h_{R}(s) and the Hilbert kernel h_{H}(s), respectively.

Starting from the classical, ramp-filter based 2D Radon inversion formula, we can develop

$$\begin{matrix}{ℛ}^{- 1} \{ p \} = {\int }_{0}^{π} {p}_{F} ( μ , {s}^{*} ) d μ \\ = \frac{1}{2 π} {\int }_{0}^{π} \frac{{\partial }^{2}}{\partial {s}^{2}} {P}_{H} ( θ , s ) {\vert}_{s = {s}^{*}} d μ\end{matrix} \qquad ( 12 )$$

with s*=u cos μ+v sin μ and with P_{H} denoting the antiderivative of p_{H} in s:

P_{H}(μ,s)=∫_{-∞}^{s}p_{H}(μ,s′)ds′+C (13)

In this derivation, we used the facts that (∂/∂s)P_{H}(μ,s)=p_{H}(μ,s) and that (∂/∂S)p_{H}(μ,s)=2πp_{F}(μ,s). Differentiating parts of the integrand in (12) twice with respect to u yields

$$\frac{{\partial }^{2}}{\partial {u}^{2}} {P}_{H} ( μ , s ) {\vert}_{s = {s}^{*}} = \frac{{\partial }^{2}}{\partial {s}^{2}} {P}_{H} ( μ , s ) {\vert}_{s = {s}^{*}} {cos}^{2} μ \qquad ( 14 )$$

and the corresponding operation with respect to v gives

$$\frac{{\partial }^{2}}{\partial {v}^{2}} {P}_{H} ( μ , s ) {\vert}_{s = {s}^{*}} = \frac{{\partial }^{2}}{\partial {s}^{2}} {P}_{H} ( μ , s ) {\vert}_{s = {s}^{*}} {sin}^{2} μ . \qquad ( 15 )$$

The combination of these two findings (14) and (15) yields

$$( \frac{{\partial }^{2}}{\partial {u}^{2}} + \frac{{\partial }^{2}}{\partial {v}^{2}} ) {P}_{H} ( μ , {s}^{*} ) = \frac{{\partial }^{2}}{\partial {s}^{2}} {P}_{H} ( μ , s ) {\vert}_{s = {s}^{*}} , \qquad ( 16 )$$

which can be substituted into (12), to finally obtain

$${ℛ}^{- 1} \{ p \} = ( \frac{{\partial }^{2}}{\partial {u}^{2}} + \frac{{\partial }^{2}}{\partial {v}^{2}} ) {\int }_{0}^{π} \frac{1}{2 π} {\int }_{- ∞}^{{s}^{*}} {p}_{H} ( μ , {s}^{′} ) d {s}^{′} d μ . \qquad ( 17 )$$

According to (17), the inverse of the 2D Radon transform of p(μ, s) can thus be obtained through: (i) Hilbert filtering of p in s, (ii) computing the antiderivative in s of this filter result, (iii) backprojecting this antiderivative into the image domain and (iv) computing the 2D Laplacian of the backprojection image.

### C. 3D Cone-Beam Reconstruction Formula

Using the findings described above, we substitute (17) into (10) and then simplify and rearrange the resulting filter operation from section A. Modification of Filter Operation. Note in this context that the two Hilbert convolutions in R⁻¹ and in (9) cancel each other and that similarly, the computation of the antiderivative cancels the differentiation in (7). Note also that the 2D Laplace operator in (17) can already be applied before computing the 2D Radon transform in step 1 of section A. Modification of Filter Operation, i.e., directly on g₁. The application of the resulting filter operation together with equations (2) and (4) then yields the new CB reconstruction algorithm, which can be written as:

### Step 1: Cosine- and Parker-Like Weighting as in (4)

$${g}_{1} ( λ , u , v ) = \frac{Dm ( λ , u )}{\sqrt{{D}^{2} + {u}^{2} + {v}^{2}}} g ( λ , u , v ) . \qquad ( 18 )$$

### Step 2: 2D Laplace Filtering (Local Operation)

$${g}_{2} ( λ , u , v ) = ( \frac{{\partial }^{2}}{\partial {u}^{2}} + \frac{{\partial }^{2}}{\partial {v}^{2}} ) {g}_{1} ( λ , u , v ) \qquad ( 19 )$$

### Step 3: 2D Radon-Based Filtering (Global Operation)

$${g}_{F} ( λ , u , v ) = \frac{1}{4 {π}^{2}} \frac{R}{D} {\int }_{0}^{π} \vert cos μ \vert {g}_{3} ( λ , {s}^{*} ) d μ \qquad ( 20 )$$

where s*=u cos μ+v sin μ and where

$${g}_{3} ( λ , μ , v ) = {\int \int }_{\underline{u} ∈ {Ω}_{λ}} {g}_{2} ( λ , u , v ) δ ( \underline{u} \cdot ( \begin{matrix}cos μ \\ sin μ\end{matrix} ) - s ) d \underline{u} \qquad ( 21 )$$

### Step 4: 3D Cone-Beam Backprojection

$${f}^{( LP )} ( \underline{x} ) = {\int }_{{λ}_{1}}^{{λ}_{2}} \frac{RD}{{[ R - \underline{x} \cdot {\underline{e}}_{w} ( λ ) ]}^{2}} {g}_{F} ( λ , {u}^{*} , {v}^{*} ) d λ . \qquad ( 22 )$$

Note that ƒ^{(FDK)} and ƒ^{(LP)} are mathematically identical for non-truncated CB data but that the algorithm disclosed herein and Feldkamp's method perform differently for reconstruction from truncated data. An intuitive reason for that is that the filtering operation in the FBP formula disclosed herein includes a purely local and a subsequent global component. The local component in (19) produces the intermediate function g₂ that can be obtained accurately for all points in D, even in presence of data truncation. Data truncation thus only affects the integration in (21) that can then only be evaluated over uεD. yielding an approximate filtered projection g_{F}^{(LP)}. Note. however, that the values of g₂ are by construction much closer distributed around 0 in most regions throughout Ω_{λ} than the values of g₁. It can thus be hypothesized that the restriction of the integration domain in (21) does not significantly change the outcome of the total filter operation.

### D. Numerical Evaluation

This section discusses the results of evaluation of the reconstruction algorithm disclosed in section C. 3D Cone-beam Reconstruction Formula, including performance comparison to the Feldkamp method. All reconstructions in this section are based on simulated CB data of the FORBILD head phantom to which noise was added while assuming an emission of 200000 X-ray photons per ray. In Experiment A. reconstruction was carried out from non-truncated data using a circular short-scan. In Experiment B, reconstruction was carried out from full-scan CB data that was truncated at all detector boundaries in each projection image. Note that both experiments involved a scan radius R=750 mm and a square detector that was visually placed at the z-axis, so that D=750 mm; additional simulation parameters are listed below in Table 1. The Feldkamp reconstruction were obtained using a smooth kernel with Gaussian apodization. The method disclosed herein was implemented using the discretization Δμ=0.25° and Δs=0.3 mm (in Experiment A) or AΔ=0.15 mm (in Experiment B), respectively.

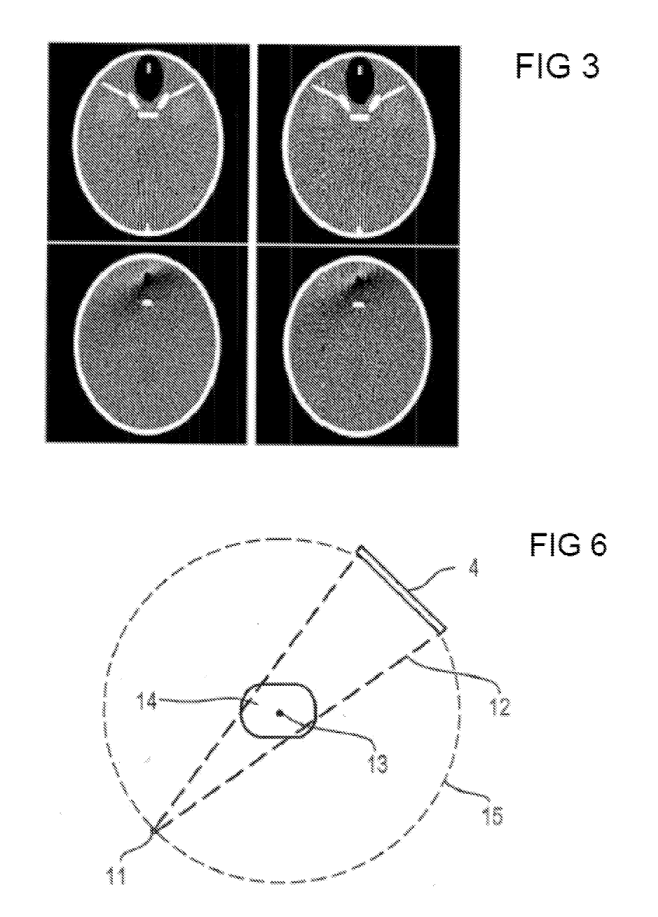

FIG. 3 presents the results of experiment A, which indicate that the both methods perform very similar for reconstruction from non-truncated data. In particular, FIG. 3 illustrates results of an example Experiment A, showing slices through a short-scan reconstruction (240 degrees) obtained from non-truncated CB data using Feldkamp (left) and the method disclosed herein (right), visualized in the window [0, 100] HU. (Top images: slice z=0 mm; bottom images: slice z=30 mm). Visually, we observe no significant difference in the noise-resolution behavior and also no difference in terms of CB artifacts.

TABLE 1
GEOMETRICAL PARAMETERS

| | Exp. A | Exp. B |
|---|---|---|
| Scan interval | 240 | 360 |
| angular increment | 1 | 1 |
| detector pixel size Δu = Δv | 0.7 mm | 0.35 mm |
| detector size uₘ = vₘ | 154 mm | 49 mm |
| reconstructed voxel size Δx = Δy | 0.5 mm | 0.5 mm |

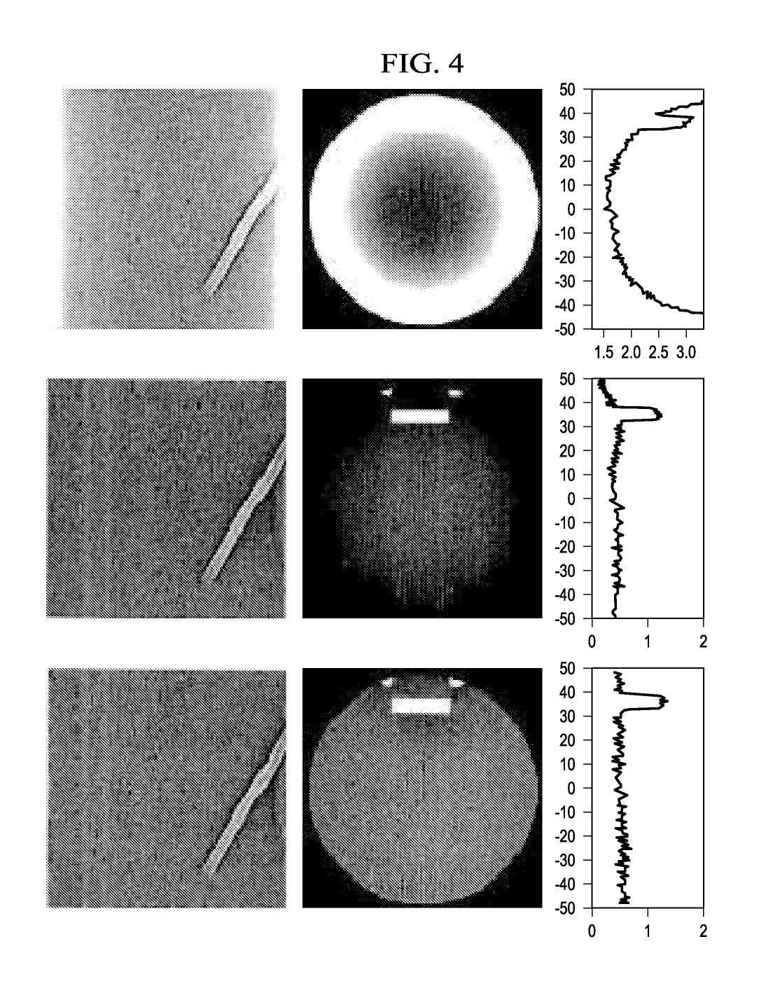

FIG. 4 displays the results of Experiment B; it shows the filtered projections g_{F}^{(LP)} and g_{F}^{(FDK)} for λ=0 next to the reconstructed slice z=0 mm. In particular, FIG. 4 illustrates reconstruction from severally truncated, full-scan CB data, for Feldkamp (top), Feldkamp with constant extrapolation (center), and the method disclosed herein (bottom). The filtered projection images at λ=0 and with uεD (left) and the reconstruction slices at z=0 mm in the window of width 800 units on the Hounsfield scale (right).

Thus, for the sake of comparison with the present method, FIG. 4 presents the results obtained with the Feldkamp method after constant extrapolation in u of CB data along each truncated detector line. We observe that among the 3 described approaches, the method disclosed herein handles data truncation in the most robust way, because it allows us to recover nicely the homogeneous background region inside the phantom. The Feldkamp methods, in contrast, clearly show truncation artifacts appearing as a radial gradient in the presented axial slices.

Thus, disclosed above is a novel FBP reconstruction algorithm for the circular CB geometry that includes the application of the 2D Laplace transform on the projection images as the first filtering operation. From non-truncated short-scan and full-scan data, the algorithm yields results that are very similar to those obtained with the Feldkamp method. An important feature of the algorithm disclosed herein is that it was designed to yield desirable reconstructions from transaxially truncated data without the need of using explicit data extrapolation schemes. Experiments using the FORBILD head phantom indicate that the algorithm yields desirable reconstruction results and may even outperform the Feldkamp method with constant data extrapolation. The algorithm may be used in various applications, e.g., for region-of-interest tomography applications in the medical or industrial field, e.g., as discussed below.

### E. X-Ray System

For diagnostic examination purposes and for interventional procedures in for example cardiology, radiology and neurosurgery, interventional X-ray systems are used for imaging, the typical essential features of which systems can be a C-arm on which an X-ray tube and an X-ray detector are mounted, a patient positioning table, a high-voltage generator for generating the tube voltage, a system control unit, and an imaging system including at least one monitor.

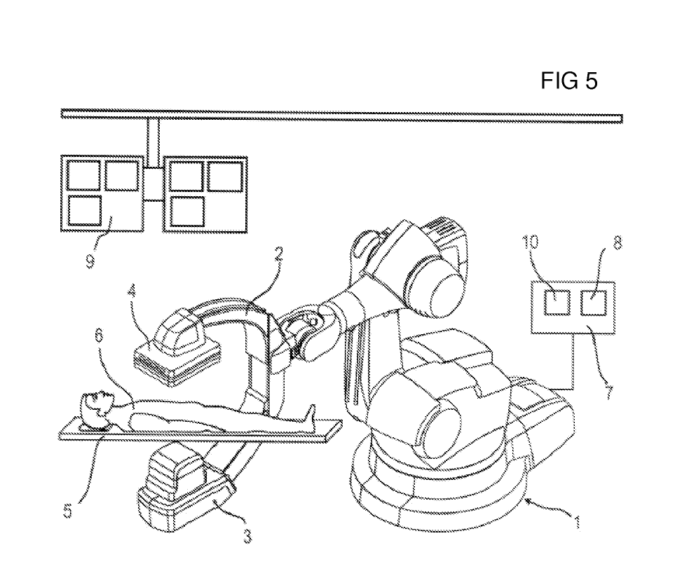

FIG. 5 illustrates an example C-arm interventional X-ray imaging system configured or programmed to implement the algorithms and methods disclosed herein, according to one embodiment. The example system may include a C-arm 2 which is rotatably mounted on a stand in the form of a six-axis industrial or articulated-arm robot 1 and at the ends of which are mounted an X-ray radiation source, for example an X-ray tube assembly 3 with X-ray tube and collimator, and an X-ray image detector 4 as image acquisition unit.

The articulated-arm robot 1, which may have six axes of rotation and hence six degrees of freedom, may enable the C-arm 2 to be moved to an arbitrary position in space, for example by being rotated around a center of rotation between the X-ray tube assembly 3 and the X-ray detector 4. The X-ray system can be rotated in particular around centers of rotation and axes of rotation in the C-arm plane of the X-ray image detector 4, e.g., around the center point of the X-ray image detector 4 and around axes of rotation intersecting the center point of the X-ray image detector 4.

The known articulated-arm robot 1 may include a base frame permanently installed on a floor, for example. A carousel may be attached to the robot, which carousel may be rotatable about a first axis of rotation. A robot rocker may be mounted on the carousel so as to be pivotable about a second axis of rotation. A robot arm that is rotatable about a third axis of rotation may be attached to the rocker. A robot hand that is rotatable about a fourth axis of rotation may be mounted at the end of the robot arm. The robot hand may include a retaining element for the C-arm 2, the retaining element being pivotable about a fifth axis of rotation and rotatable about a sixth axis of rotation running perpendicular thereto.

The X-ray image detector 4 can be a rectangular or square, flat semiconductor detector which may be produced from amorphous silicon (a-Si), for example. Integrating or counting CMOS detectors can also be used, however.

A patient 6 to be examined is placed as the examination subject in the beam path of the X-ray tube assembly 3 on a patient positioning table 5 so that images of the heart, for example, can be recorded. Connected to the X-ray diagnostic apparatus is a system control unit 7 having an imaging system 8 and a processing circuit 10 that process the image signals from the X-ray image detector 4 (control elements are not shown, for example). In one embodiment, X-ray detector 4 may collect cone-based (CB) projection data, and pass such data (directly or indirectly, e.g., for intermediate processing) to processing circuit 10. The processing circuit 10 may be configured to perform a tomographic reconstruction to generate X-ray images as discussed above, e.g., by a process including receiving the CB projection data associated with the object from the X-ray detector 4, applying a weighting function to the received data, performing a localized filtering of the data using a 2D Laplace operation, performing a non-localized filtering of the data using a 2D Radon-based filtering operation, and performing a 3D Cone-beam backprojection of the data to generate a tomographic image. The X-ray images can then be viewed on a monitor 9.

The X-ray tube assembly 3 emits a bundle of rays 12 originating from a beam focus 11 of its X-ray radiation source and striking the X-ray image detector 4. If it is intended to generate 3D data sets according to the so-called DynaCT method for low-contrast visualization of for example soft tissue, the rotatably mounted C-arm 2 with X-ray tube assembly 3 and X-ray image detector 4 may be rotated in such a way that, as FIG. 6 shows schematically in a view onto the axis 13 of rotation, the X-ray tube assembly 3—represented figuratively here by its beam focus 11 as well as the X-ray image detector 4—moves around an examination subject 14 located in the beam path of the X-ray tube assembly 3 on an orbit 15. The orbit 15 can be traversed completely or partly for the purpose of generating a 3D data set.

In this case the C-arm 2 with X-ray tube assembly 3 and X-ray image detector 4 may move according to the DynaCT method, e.g., through an angular range of at least 180°, for example 180° plus fan angle, and record projection images in rapid succession from different projections. The reconstruction can be carried out based on just a subset of said acquired data, e.g., using the method discussed above. The subject 14 to be examined can be for example an animal or human body or indeed a phantom body.

The X-ray tube assembly 3 and the X-ray image detector 4 each rotate about the object 5 in such a way that the X-ray tube assembly 3 and the X-ray image detector 4 are disposed on opposite sides of the subject 14.

In normal radiography or fluoroscopy by means of an X-ray diagnostic apparatus of this type the medical 2D data of the X-ray image detector 4 may be buffered in the imaging system 8 if necessary and subsequently displayed on the monitor 9.

What is claimed is:

1. A method for performing a 3D tomographic image reconstruction in the circular geometry, comprising:
a processor receiving cone-based (CB) projection data associated with an object;
the processor applying a weighting function to the received data;
the processor performing a localized filtering of the data using a 2D Laplace operation;
the processor performing a non-localized filtering of the data using a 2D Radon-based filtering operation; and
the processor performing a 3D Cone-beam backprojection of the data to generate a tomographic image.

2. A system for performing a 3D tomographic image reconstruction in the circular geometry, comprising:
an X-ray source;
an X-ray detector configured to detect radiation from the X-ray source to collect cone-based (CB) projection data associated with an object; and
a processing circuit configured to generate a tomographic image of the object by:
applying a weighting function to the received data;
performing a localized filtering of the data using a 2D Laplace operation;
performing a non-localized filtering of the data using a 2D Radon-based filtering operation; and
performing a 3D Cone-beam backprojection of the data to generate the tomographic image of the object.
